(19) United States
(12) Patent Application Publication
(10) Pub. No.: US 2026/0276572 A1
(43) Pub. Date: Sep. 17, 2026

(54) In-Situ Measurement of Gas Entrainment in a Multi-Phase Fluid

(71) Applicant: Southwest Research Institute, San Antonio, TX (US)

(72) Inventors: Marshall C. Hudson, San Antonio, TX (US); Nolan Erickson, Glen Allen, VA (US); Francisco A. Espinal, San Antonio, TX (US)

(21) Appl. No.: 19/567,221

(22) Filed: Mar. 15, 2026

Related U.S. Application Data
(60) Provisional application No. 63/773,031, filed on Mar. 17, 2025.

(51) Int. Cl. G01N 27/22; G01N 33/28

(52) U.S. Cl. CPC G01N 27/221; G01N 33/2823; G01N 2027/222

(57) ABSTRACT

A method of measuring gas entrainment of a gas within a multi-phase fluid (a gas within a liquid) contained in a passageway, vessel, or other container. An open-ended coaxial probe is installed though a wall of the passageway such that an open end of the probe is flush or nearly flush with the inner wall. The probe is then used to measure permittivity of the multi-phase fluid. The volume fraction of the gas within the fluid is calculated, based on a mixing function that expresses permittivity as a function of the pure gas permittivity and the pure liquid permittivity.

### BACKGROUND OF THE INVENTION

“Aeration of a lubricant” means the process of air bubbles becoming trapped within a lubricating oil or other lubricating substance. Air is mixed into the oil, such as by agitation or turbulence within the lubrication system. Aeration can negatively affect a lubricant's durability and ability to act as a heat transfer fluid, hydraulic working fluid, rust preventative agent, wear preventative agent, or friction reduction agent.

Measuring aeration of a lubricant is important because air trapped within the lubricant can impact its performance and hence the performance of the lubricated equipment.

Aeration of a lubricant can be measured by sampling or by in-situ methods. In situ measurement is a process of measuring the amount of air entrapped within a lubricant while it is within a system. Various methods of in-situ measurement include using ultrasonic sensors, capacitive sensing, or optical methods to monitor the air bubbles present in the fluid flow, without needing to extract a sample first.

In the automotive industry, conventional lubricant aeration measurement systems consist of a separate measurement circuit in which lubricant is drawn from the sump or pressurized gallery and pumped through an aeration measurement device or densitometer to quantify the entrained air content. Operation of this measurement system adds fluid volume and complexity to the system and can change the local entrained air fraction from the “true” value in the hardware prior to the aerated fluid reaching the sensor.

Aeration of a lubricant is but one example of gas entrainment that can cause operational issues in various applications. In any fluid flow, a gas (such as air, nitrogen, or vapor) can be drawn into a liquid stream, creating a multi-phase flow. For example, gas entrainment can cause reduced pump efficiency, inaccurate flow measurements, and equipment damage.

### BRIEF DESCRIPTION OF DRAWINGS

FIG. 1 illustrates the gas entrainment measurement probe used in an automotive lubrication system.

FIG. 2 illustrates the active end of the probe, with the probe installed in the wall of a passageway.

FIG. 3 illustrates the electric field in the lubricant as the probe is activated.

FIGS. 4-6 illustrate various embodiments of the probe tip.

### DETAILED DESCRIPTION OF THE INVENTION

The following description is directed to using an open-ended coaxial probe to measure gas entrainment. For purposes of example, the description is primarily in terms of aeration experienced by a lubricant. The tip of the probe is exposed to the lubricant and excited by high-frequency AC signals. One or more dielectric properties are then measured. These dielectric properties are known to change with entrained air content, so aeration may be calculated from the measured properties and data collected from prior calibration measurements.

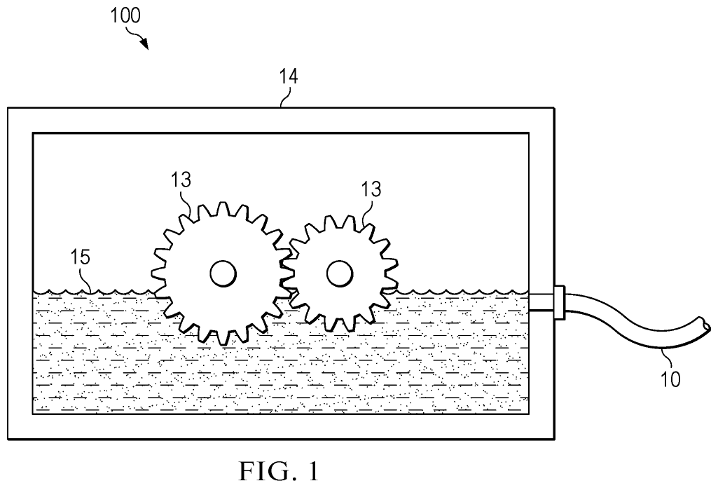

FIG. 1 illustrates a gear box 100 of a vehicle engine, the gear box 100 having lubricated gears 13 in a gearbox case 14 that contains the lubricant 15. An in-situ aeration measurement probe 10 is installed where it accesses lubricant 15 within gearbox 100. This is one example of an automotive application of probe 10, which could be placed anywhere within a vehicular lubrication system, such as in the sump, or in the lubricant flow path (gallery) or elsewhere in automotive hardware, such as in the transmission, transaxle, axle, electric drive unit, engine, or other locations. In other embodiments of the use of probe 10 for lubrication systems, probe 10 may be used in any passageway or vessel.

The use of probe 10 to measure aeration of a lubrication system is but one example of the use of probe 10 to measure gas entrainment. As indicated in the Background, gas entrainment can be of interest in many different applications. The entrained liquid can be other than a lubricant and the entraining gas can be other than air. As an example, in oil and gas pipelines, the fluids can contain a wide spectrum of hydrocarbon “weights”, such as natural gas bubbles. For purposes of this description, various structures containing entrained (multi-phase) fluids are collectively referred to herein as “passageways”.

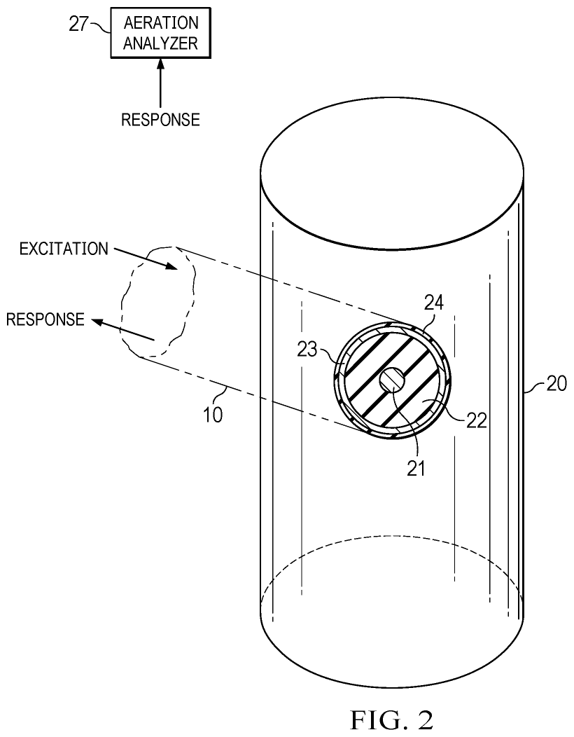

FIG. 2 illustrates the active end of probe 10, as installed through the wall of a passageway 20 containing a lubricant. Probe 10, more specifically described as an open-ended coaxial probe, is a broadband sensor used to measure dielectric properties of materials through the analysis of reflected electromagnetic waves. It comprises a central electrode 21 surrounded radially by an insulator 22, metallic shield 23, and outer jacket 24. This may be the same construction as a conventional coaxial cable that has been cross-sectionally cut but not otherwise terminated.

The open end of probe 10 is placed such that its exposed end is exposed to the fluid lubricant to be measured. Probe 100 can be very small in diameter and installed such that it is flush or nearly flush with the inner wall of the vessel or pipe, such as passageway 20, containing the fluid lubricant. As a result, probe 100 has a minimal impact on fluid dynamics and/or pressure drops along the lubricant's flow path and has no geometry to trap or restrict movement of air bubbles.

For automotive applications specifically, probe 10 is small enough to fit within areas of interest in automotive hardware (transmission, transaxle, axle, electric drive unit, engine, etc) and is capable of measuring the entrained air fraction in-situ. It is sufficiently small so as to not significantly impact fluid dynamics or pressure within the test area and allows free interaction of air bubbles and fluid within its sensing volume without trapping or restricting bubble movement.

As stated in the Background, aeration is known to affect other, measurable bulk properties of a composite fluid. Conventional aeration measurement devices function by correlating a measurement of a bulk property of the composite fluid to measurements of that property taken previously for the pure liquid and pure gas phases of the composite fluid.

In the case of probe 10, the properties of interest are AC dielectric properties. The operating principle of probe 10 is based on propagating a guided wave along its coaxial transmission line and observing the reflection that occurs when the wave reaches the open termination at the interface between probe 10 and the lubricant of interest.

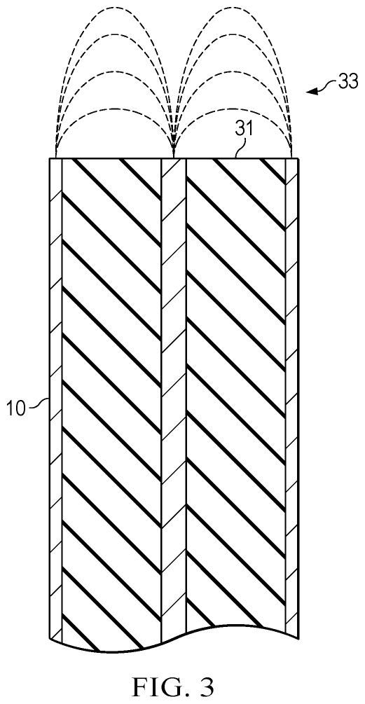

As illustrated in FIG. 3, at the open end (aperture) 31 of probe 10 when excited with high frequency AC signals, the electric fields 33 are no longer fully confined within the coaxial dielectric and electric field lines fringe into the surrounding lubricant. The distribution of these fringing fields 33 depends on the dielectric properties of the material under test. By measuring the reflection coefficient at the feed of probe 10, it is possible to infer the dielectric properties of the aerated lubricant (a dielectric fluid) in contact with the probe's aperture.

Referring again to FIG. 2, a system for measuring aeration (or other gas entrainment) includes both probe 10 and an analyzer processor 27. Processor 27 has appropriate hardware and programming for receiving response signals from probe 10 and performing the tasks described herein.

As stated above, probe 10 may be very small and the sensing volume extends past the tip of probe 10 into the fluid, meaning it need only be flush with the inside wall of the passageway containing the lubricant. As a result, probe 10 has minimal impact on fluid dynamics and/or pressure drops along the lubricant flow path and no geometry to trap or restrict movement of air bubbles, which would negatively affect measurement accuracy. Its small size means it can be installed where conventional sensors cannot so that aeration may be measured in-situ. This eliminates “observer effects” in the measurement of aeration which would otherwise stem from using large sensors or performing the measurement in a separate measurement stream.

For aeration measurement, the dielectric property measurements are transformed into aeration estimates using data collected from a preliminary calibration process performed on the fluid of interest prior to testing. Using relative permittivity (ϵᵣ) as an example of a dielectric property, ϵᵣ of the pure gas phase is measured. For this example, this is assumed to be a constant relative to temperature and other environmental conditions, but it can be measured at a variety of temperatures, humidities, concentrations, etc. to fully characterize the gas's permittivity under a variety of conditions provided these conditions are known or measured at the aeration measurement site during testing. Similarly, the ϵᵣ of the pure liquid phase is measured, typically at a spectrum of temperatures covering the temperature range expected during testing. It is critical, at a minimum, that temperature and pressure are known or measured near the aeration measurement site. In the example of aeration of a lubricant, the above-described calibration would be the measurement of relative permittivity for pure air and then for pure lubricant.

This calibration data can be performed prior to actual testing and the data stored in analyzer 27. In operation, probe 10 is used in-situ to measure the permittivity of the composite fluid.

A mixing rule is required for the dielectric property of interest. This allows determination of the effective, measurable properties of the fluid of interest, based on the individual dielectric properties and volume fractions of its components.

For permittivity, one such mixing rule found in literature (Landau-Lifshitz mixing rule) indicates that the permittivity of a mixture may be expressed as a function of the permittivitys of the components as follows:

$$\sqrt[3]{{ϵ}_{mix}} = α \sqrt[3]{{ϵ}_{gas}} + ( 1 - α ) \sqrt[3]{{ϵ}_{fluid}} ,$$

where ϵₘᵢₓ is permittivity of the composite fluid, ϵ_{gas} is permittivity of the pure gas phase, ϵ_{fluid} is permittivity of the pure liquid phase, and a is the volume fraction of the gas phase in the mixture.

Thus, if the permittivity of the gas is a constant, the permittivity of the liquid is a function of temperature, the temperature at the aeration measurement site is known or measured, and the permittivity is measured at the same location, then the volume fraction of the gas phase is equivalent to aeration of the sample and may be calculated as:

$$α = \frac{\sqrt[3]{{ϵ}_{mix}} - \sqrt[3]{{ϵ}_{fluid} ( T )}}{\sqrt[3]{{ϵ}_{gas}} - \sqrt[3]{{ϵ}_{fluid} ( T )}}$$

The volume of a discrete mass of air (bubble) is proportional to the static pressure. Thus, if the static pressure at the measurement site is also known or measured, then an equivalent volume fraction of air (aeration) may be calculated for any other pressure (often for a standard pressure of 1 atmosphere).

As stated above, probe 10 and its method of use described herein can be used for various situations in which a fluid becomes entrained. The fluid must be sufficiently dielectric to provide the dielectric measurements described herein and may be thus referred to herein as a “dielectric fluid”. The fluid may comprise a liquid having bubbles of any composition, provided that the fluid in the bubbles has sufficiently different dielectric properties to the liquid so that the concentration of gas can be correlated with its measured dielectric properties.

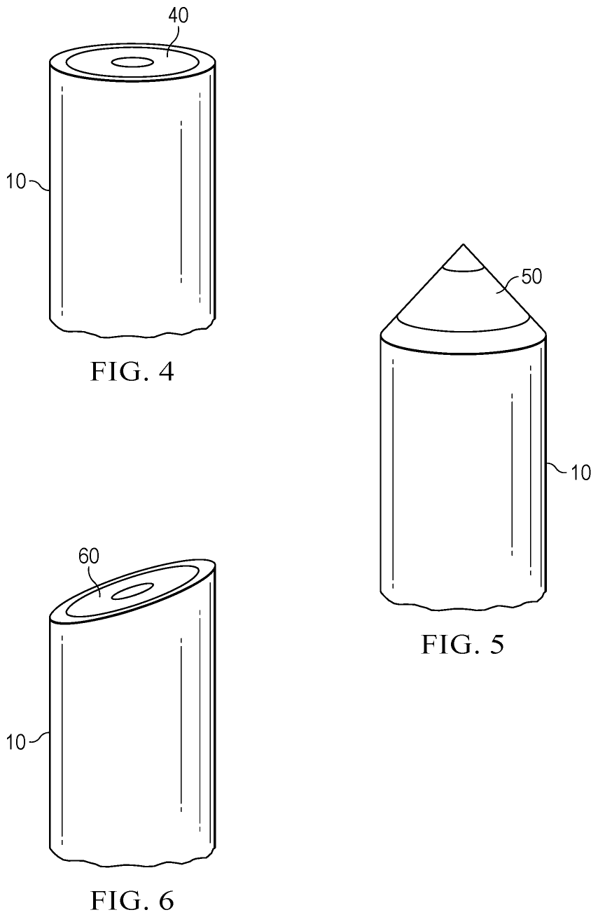

FIGS. 4-6 illustrate various embodiments for the tip of probe 10. FIG. 4 illustrates a probe 10 having a flat tip 40. Other probe tips may be deliberately designed to increase their sensing depth or aim the fringing field in a specific direction for improved measurement accuracy along that axis by modifying the probe tip geometry. FIG. 5 illustrates a conical tip 50, and FIG. 6 illustrates an oblique tip 60. As stated above, each of these probe tips allows probe 10 be to installed flush or nearly with the inner wall of a lubricant passageway, such that only the flat, conical, or oblique tip is exposed to the lubricant.

1. A method of measuring gas entrainment of a gas within a multi-phase fluid (a gas within a liquid) contained in a passageway, both the gas and the liquid having measurable permittivity, comprising:
measuring the permittivity of the gas in a pure gas state and the permittivity of the liquid in a pure liquid state, thereby obtaining a pure gas permittivity value and a pure liquid permittivity value;
installing a coaxial probe though a wall of the passageway such that an open end of the probe is flush or nearly flush with the inner wall of the passageway;
using the coaxial probe to measure permittivity of the composite multi-phase fluid; and
calculating the volume fraction of the gas within the multi-phase fluid, based on a mixing function that expresses permittivity as a function of the pure gas permittivity value and the pure liquid permittivity value.

2. The method of claim 1, wherein the gas is air.

3. The method of claim 1, wherein the liquid is a lubricant.

4. The method of claim 1, wherein the permittivity is relative permittivity.

5. The method of claim 1, wherein the probe tip is flat.

6. The method of claim 1, wherein the probe tip is oblique.

7. The method of claim 1, wherein the probe tip is conical.

8. The method of claim 1, further comprising measuring the pressure of the fluid at the measurement site and calculating the volume fraction of the gas at other pressures.

9. The method of claim 1, further comprising adjusting the pure gas permittivity data for temperature and/or pressure at the measurement site.

10. The method of claim 1, further comprising adjusting the pure liquid permittivity data for temperature and/or pressure at the measurement site.

11. A system for measuring gas entrainment of a gas within a multi-phase fluid (a gas within a liquid) contained in a passageway, both the gas and the liquid having measurable permittivity, comprising:
an analyzer processor that stores values representing the permittivity of the gas in a pure gas state (a pure gas permittivity value) and the permittivity of the liquid in a pure liquid state (a pure liquid permittivity value);
an open-ended coaxial probe installed though a wall of the passageway such that an open end of the probe is flush or nearly flush with the inner wall of the passageway;
wherein the analyzer processor is further operable to receive data from the probe representing the permittivity of the composite multi-phase fluid, and to calculate the volume fraction of the gas within the multi-phase fluid, based on a mixing function that expresses permittivity as a function of the pure gas permittivity value and the pure liquid permittivity value.

12. The system of claim 11, wherein the gas is air.

13. The system of claim 11, wherein the gas is air.

14. The system of claim 11, wherein the liquid is a lubricant.

15. The system of claim 11, wherein the permittivity is relative permittivity.

16. The system of claim 11, wherein the probe tip is flat.

17. The system of claim 11, wherein the probe tip is oblique.

18. The system of claim 1, wherein the probe tip is conical.
